(19) United States
(12) Patent Application Publication
(10) Pub. No.: US 2022/0324961 A1
(43) Pub. Date: Oct. 13, 2022

(54) ALLERGY THERAPY

(71) Applicant: IgGenix, Inc., South San Francisco, CA (US)

(72) Inventor: Kari Nadeau, South San Francisco, CA (US)

(21) Appl. No.: 17/541,854

(22) Filed: Dec. 3, 2021

Related U.S. Application Data
(60) Provisional application No. 63/173,762, filed on Apr. 12, 2021.

(51) Int. Cl. C07K 16/24; G01N 33/68; C07K 16/28

(52) U.S. Cl. CPC C07K 16/247; G01N 33/6893; C07K 16/244; C07K 16/2866; C07K 2317/33; G01N 2800/24

(57) ABSTRACT

The invention provides methods and compositions for treating allergies that use antibodies or other products of the immune system from whom have already experienced and responded to an allergen. In particular, people who have suffered from an allergic reaction to an allergen but subsequently become desensitized to the allergen produce immune products that may be isolated or reproduced for use in a therapeutic composition.

### TECHNICAL FIELD

The disclosure relates to the treatment of allergies.

### BACKGROUND

The immune system relies on functionally-specialized cells. One such cell type, the lymphocytes, are of primary importance in the adaptive immune system. The main lymphocyte types are the B cells and T cells, which are produced from hematopoietic stem cells in bone marrow. B cells and T cells appear identical until they are activated. T cell progenitors travel to, and mature in, the thymus, while B cells mature in the bone marrow. Specifically, naïve T cells are those that have yet to encounter an antigen, but then differentiate when presented in the lymph nodes with an antigen by an antigen presenting cell (APC).

In the body, the peripheral lymphoid organs, which include the lymph nodes, spleen, tonsils, and mucosal-associated lymphoid tissue (MALT), typically contain B and T cells at varying stages of differentiation: naive B and naive T cells that have left the bone marrow or thymus but have yet to encounter their matching antigen, effector cells that have been activated by their matching antigen, and memory cells from past infections.

Part of the adaptive immune response is the antibody response carried out by the B cells. In an antibody response, activated B cells secrete antibodies, which travel through the bloodstream and bind to “foreign” materials, such as pathogens, and block their action directly or mark them for destruction by the immune system. Sometimes, however, the adaptive immune system reacts to harmless substances in the environment, resulting in an allergic response. Common allergies include food allergies, hay fever, and asthma. Certain allergens may produce a systemic allergic response that may manifest as skin reactions, bronchoconstriction, swelling, low blood pressure, coma, and even death. Allergies, especially food allergies, continue to be a threat to public health and additional, more effective treatments and prophylactics are needed.

### SUMMARY

The invention provides methods and compositions for treating allergies using antibodies or other products of the immune system obtained from people who have already experienced and responded to a target allergen. For example, when a person has become desensitized to an allergen, that person's blood, plasma, or serum may be source of factors, such as cellular or molecular species that neutralize the allergen, and inhibit its ability to trigger an allergic or anaphylactic response. Those factors are obtained, isolated, and/or identified and enriched, formulated, or included in a composition that is useful as a therapeutic to treat allergies. For example, person who has become desensitized to an allergen may be desensitized because their immune system produces an immunoglobin, such as an IgE or IgG4, or other immune-cell product that inhibits the natural allergic reaction. Those immune-cell products are obtained from the desensitized subjects and used in therapeutic compositions to treat people to protect them against allergies. In a treated person, the immune-cell product obtained from the desensitized subject operates to protect against an allergic reaction, for example, by binding to an allergen and blocking it from binding to IgE and triggering an allergic reaction.

It is understood that the anaphylactic response arises when effector B cells of a person's immune system produce IgE immunoglobin specific to an allergen. In that person, an Fc portion of the IgE molecules bind to Fc receptors on mast cells and basophils. When those IgE molecule encounter the allergen again, those allergens bind to, and cross-link, the IgE-receptor complexes on the mast cells and basophils. That cross-linking on mast cells and basophils is what leads to a process called degranulation, in which those cells release histamine and inflammatory mediators, which then leads to an anaphylactic response. However, people who have become desensitized to an allergen are suspected to produce products that interfere with degranulation by, for example, preventing allergen from binding to, and cross-linking, IgE-receptor complexes on mast cells and basophils. Those immune products that inhibit degranulation may primarily include IgG4 immunoglobins that are produced by the desensitized person's immune system and that specifically bind to allergen but are not capable of cross-linking Fc Epsilon receptors, but can bind Fc gamma receptors and cause inhibition.

The immune system of a desensitized patient may produce products other than IgG4 that inhibit anaphylaxis. For example, the desensitized immune system may produce other immunoglobulins, or other molecular species that bind to, and interfere with, other molecules or cells that promote the allergic response. For example, desensitization may involve an antibody that binds to a cytokine (such as, for example, IL-4R, IL-4, IL-13, IL-33, IL-9, and IL-5) that promotes the allergic response. In other examples, desensitization may involve an IgE or a fragment of an IgE that is specific to an allergen but is not competent for binding and cross-linking an Fc receptor. In another example, desensitization may arise if the immune system promotes or produces regulatory T cells (Treg cells) or helper T cells 1 (Th1 cells).

The invention involves identifying cells or isolating their products from the immune system of a desensitized person and using those products in a therapeutic for the treatment or prevention of allergic reactions. The products in the therapeutic are used to inhibit degranulation or other components of the allergic response. Those products may primarily include IgG4 immunoglobins that are produced by the desensitized person's immune system. Moreover, the antigen-binding portion of the immunoglobin—the paratope—may have greater binding specificity for an epitope of an allergen. Even more importantly, the paratope of an IgG4 may have specificity for an epitope of the allergen that was already specifically recognized by an IgE of the person or even bind to more epitopes of an allergen due to the process of desensitization. An insight of the disclosure is that the desensitized immune system has already performed a version of a natural “epitope discovery” assay for the discovery of the epitope implicated in anaphylaxis or its cognate paratope on an antibody, discovered during desensitization.

The literature reports that common allergens may each have some number of different epitopes and that numerous laboratory assays are used for epitope discovery (see, e.g., Matsuo, 2015, Allergology Int 64(4):332-343, incorporated by reference). A desensitized person may present a ready supply of antibodies (e.g., IgG4) with a paratope specific for the epitope implicated in the allergy. By isolating, sequencing, modeling, or cloning those antibodies or other related anti-anaphylactic cell or molecular species, those immune products may be used in allergy therapies.

In certain illustrative embodiments, IgG4 from a desensitized individual are identified, e.g., by RNA-Seq, cloned and expanded in culture or by hybridoma technology, and harvested as a therapeutic ingredient. Because the sequence is obtained from the desensitized individual, the clonal antibody product will have a paratope specific for an allergen epitope implicated in the allergic reaction. Where samples from multiple desensitized individuals with a common allergy (e.g., peanut) is available, e.g., as a blood bank of samples from subjects successfully treated by oral immunotherapy, the IgG4s or other Ig molecules (i.e. IgG or IgA or IgM or IgD molecules) produced from those samples can provide a suite of therapeutics with a high probability of including a paratope specific to an epitope implicated in any given case of allergy.

These insights of the invention are applicable in another scenario in which an epitope implicated in allergy apparently changes over time. In particular, some individuals allergic to a particular allergen may have high levels of antibodies (e.g., of an IgE) against a specific epitope of the allergen. However, at some later time, e.g., months or years later, one of those individuals may have high levels of antibodies (e.g., of a second IgE) against a different, second epitope of the same allergen. For example, some results have suggested that oral immunotherapy may have an effect that leads to increased diversity in either or both of IgE and IgG4 (or other Ig molecule) binding, with the implication that after treatment for an allergy, a person may latter produce IgE and/or IgG4 (or other Ig molecule) specific to a second epitope that was not the epitope involved in the allergy that was initially treated. In such cases, the person's immunological products present an opportunity to therapeutically target an allergenic epitope that had not independently been discovered or recognized as causing anaphylaxis.

Therapeutic applications of this discovery involve identifying a subject who exhibits an allergic response to IgE-binding epitopes that exhibit diversity over time. Assays are performed to obtain or identify at least first and second antibodies from the subject, which antibodies are specific for respective first and second epitopes. Those assays are used to produce IgG4 or similar immune product against the second epitope, and that immune product is used for making a therapeutic treatment.

An insight of this approach is that a subject with an allergy to an allergen may change which epitopes to which he or she adversely responds. Further, that change may be hastened or promoted by allergy treatments such as oral immunotherapy. For example, a subject may become desensitized to an epitope by immunotherapy, but that very immunotherapy may increase the diversity of antibodies produced by that subject. Because of the increased diversity of antibodies, the subject may become sensitized to some second epitope that was not initially implicated in the allergy. However, that same subject may also now produce IgG4 or some other immune product specific to that second epitope.

A sample may be obtained from a subject who has an allergy to an allergen. An immune product such as an IgG4 specific to the second epitope can be identified or isolated from the sample and used in the production of medicament for treating allergy and/or preventing an anaphylactic response to the allergen. This approach is used when the epitope implicated in the allergic response apparently changes over time, and subjects are responding to different epitopes of an allergen. In such cases where a subject has an allergy and made IgE against a first epitope and now makes IgE against a second epitope, the disclosure provides methods that include identifying or isolating from the subject an immune product specific to the second epitope, such an IgG4, and using the immune product in allergy therapy.

In certain aspects, the invention provides methods of allergy treatment. The methods include obtaining sample from a person who has become desensitized to an allergy, analyzing the sample to identify a molecular species that inhibits an allergic response, preparing a therapy that includes the molecular species, and administering the therapy to a patient susceptible to the allergic response. Optionally, the molecular species is an IgG4 (or other Ig molecule) that binds an allergen.

In some embodiments, the molecular species is an IgE fragment incapable of binding and/or cross-linking an Fc receptor. Either an IgE fragment may be obtained from the person, or a full IgE may be obtained, but a fragment may be prepared for use in the therapy. E.g., methods may include determining a sequence of an IgE in the sample that promotes the allergic response and using sequence information of the IgE to create an antibody fragment for use in the therapy, wherein the antibody fragment binds an allergen but does not bind to Fc receptors and cross-link. For example, IgE sequences with modified or missing constant regions may be cloned, expressed, and collected for use in a therapeutic product. Thus, the molecular species may include a fragment of an IgE that binds to an allergen that promotes the allergic response, wherein the fragment does not bind to a high-affinity Fc receptor.

Some embodiments of the invention make use of a collection or bank of sample from patients known to have become desensitized to an allergy. Methods may include obtaining the sample from such a sample bank of, e.g., blood, serum, or plasma samples from people who have been become desensitized to allergies naturally or by therapy. A plurality of different IgG4s (or other Ig molecule) specific to an allergen may be identified or isolated from respective different samples from the sample bank.

Analyzing the sample to identify a molecular species that inhibits an allergic response may include performing assays on samples in a sample bank from people who have been become desensitized to allergies to detect molecular species that bind an allergen. Suitable samples may include blood, plasma, or serum. Suitable assays may include nucleic acid sequencing, protein binding assays, or similar. Assays may include RNA-Seq to identify IgG4 transcripts or capture assays using proteins that bind the allergen (e.g., specific capture onto beads in a column for subsequent elution and collection).

Analyzing the sample to identify a molecular species that inhibits an allergic response may be done by sequencing IgG or other Ig molecule transcripts from the sample to determine a sequence of an IgG4. The identified sequence may be used in includes creating a monoclonal antibody product comprising the IgG4. Preferably the IgG4 binds to an epitope of the allergen that is recognized by an IgE of the person being treated.

Methods may be used to address other targets that participate in the allergic response. For example, the molecular species may be an antibody that binds a cytokine that promotes the allergic response. The antibody may bind to the cytokine and inhibit the ability of the cytokine to promote the allergic response. For example, the antibody could bind to one of IL-4R, IL-4, IL-13, IL-33, IL-9, or IL-5. Other targets that are useful include cells. For example, the molecular species may include a regulatory T cell (Treg), a T helper type 1 (Th1) cell, or a fragment thereof.

Other aspects of the disclosure provide methods of treating allergy that include detecting—from a subject with an allergy—IgE specific to an epitope of an allergen, determining that B cells from the subject have produced a second IgE specific to a second epitope of the allergen, and identifying a sequence or structure of the second epitope or a paratope of the second IgE. Some embodiments involve a subject who has been treated to attenuate allergic response to a (first) epitope, and the embodiments further involve identifying the sequence of a second (later) epitope implicated in in allergic response and using the identified sequence of the second epitope to determine a therapy for use in an additional, subsequent treatment for allergic response. For example, an IgG4 or similar immune product specific to the second epitope may be isolated or cloned and used in a therapeutic. These methods involve a change over time of the specific epitope implicated in the subject's allergy. For example, a first sample from the subject may include IgE specific to the epitope and a second, subsequent, sample from the subject may include a second IgE specific to the second epitope. For such situations, methods of the invention may include preparing a therapeutic composition that prevents the second epitope from promoting IgE cross-linking in vivo. Any suitable therapeutic composition may be prepared. Preferably, the therapeutic composition includes a molecular blocker that interferes with in vivo binding of IgE to the allergen. For example, the blocker may include an IgG4 that binds the second epitope. In some embodiments, wherein the blocker comprises a molecular structure bearing one copy of the second epitope and no other epitopes of the allergen, i.e., a synthetic or modified version of the allergen that will present the second epitope for binding IgE but will be incapable of binding and cross-linking more than one Fc receptor. In certain embodiments, the blocker comprises an IgE fragment with a paratope to the second epitope, wherein the IgE fragment does not bind to an Fc receptor and cross-link.

Identifying the sequence of the second epitope may include isolating the second IgE, or transcripts for the second IgE, and performing an assay using the isolated second IgE to determine the sequence of the second epitope. Suitable assays may include RNA-Seq, crystallography, electron-microscopy, array-based screening, and mass-spectrometry and may be used to determine the sequence and/or structure of the second IgE. An important insight is that the method is applied to patients for whom the epitope implicated in the allergy has changed over time. Such patients may have undergone oral immunotherapy that was initially successful, but may later exhibit an anaphylactic reaction to some second, other epitope not addressed by the immunotherapy. For such patients, methods of the invention include adding the sequence or structure of the second epitope to a database of therapeutic targets. The change over time may recur. For example, subsequent B cells from a patient may produce a third IgE specific to a third epitope of the allergen and methods of the invention may involve identifying a sequence or structure of the third epitope or a paratope of the third IgE. Further, methods of the invention may include producing a therapeutic such as a monoclonal IgG4 specific to one of the first and second (or subsequent) epitopes. Methods may include isolating from the subject IgG4 that binds the second epitope and/or making a monoclonal IgG4 or other Ig molecules for use as a therapy against the allergy.

### DETAILED DESCRIPTION

Allergy is an adaptive immune response to non-infectious substances referred to as allergens. Common allergens include food, mites, pollen, pet dander, mold, vaccines, medicines, insect venom, and latex. Some allergic reactions, such as contact dermatitis, are thought not to involve IgE. However, many allergic responses such as anaphylaxis, allergic rhinitis (hay fever), some food allergies and allergic asthma, involve IgE and T helper 2 (TH2) cells that recognize antigenic epitopes of allergens. In IgE-mediated responses, exposure to the allergenic substance may induce IgE production and sensitize the subject. The immune system hyperproduces IgE after initial contact with an allergen. The subject will then have circulating IgE immunoglobins specific to the allergen. Once threshold levels of IgE are met, the subject is susceptible to reaction to the allergen in the future. If the subject later encounters the same allergen, they experience an allergic reaction often characterized by inflammation.

Allergen exposure produces an acute reaction, which is known as an early-phase reaction or a type I immediate hypersensitivity reaction. An IgE-mediated type I immediate hypersensitivity reaction that can occur within minutes of allergen exposure. In those cases, IgE bound to FcεRI on mast cells and basophils is crosslinked when multiple IgE/Fc complexes bind to an allergen. That cross-linking causes the mast cells and basophiles to degranulate, releasing mediators, such as histamine and cytokines. Those mediators promote vasodilation, vascular permeability, and responses such as bronchoconstriction. Some of those mediators also promote the recruitment or activation of leukocytes, contributing to the development of subsequent “late-phase” reactions, with continuing inflammation and breathing trouble, over subsequent hours to days. The allergic reaction may involve natural feedback loops. For example, when basophils and mast cells degranulate in response to contact with IgE, they release IL-4, which may promote class-switching of B cells, potentially leading to increased production of IgE.

One approach to treating allergies clinically has involved immunotherapy, often in the form of oral immunotherapy. The principle of immunotherapy is to retrain the immune system to not produce IgE upon antigen contact. Some approaches to immunotherapy involve the slow introduction of small amounts of antigen (e.g., of an allergen), which amounts may increase over time. It may be found that such allergen exposure increases the subject's production of immunoglobin IgG4 specific to the allergen. It may be theorized that the IgG4 then competes with IgE to reach the target protein. That is, when the allergen is encountered, the IgG4 binds to available epitopes of the allergen, leaving no place on the allergen for IgE to bind. Because IgE cannot bind allergen, even if IgE is bound to the Fc receptors on mast cells and basophils, nothing is available to bind the paratopes of the IgE molecules and cross-link multiple IgE-Fc receptor complexes. The presence of the IgG4 prevents the IgE/Fc complexes from being cross-linked, the IgG4 prevents mast cells and basophils from degranulating, and thus inhibits anaphylaxis.

Targets for allergy therapy may be recognized by understanding the cellular and molecular effects of allergy and of immunotherapy. In one set of likely effects of immunotherapy, the allergen delivered therapeutically is carried by antigen presenting cells to lymph nodes, activating T cells and leading to the production of Tregs. Tregs suppress Th2 and stimulate growth of Th1 cells. Interaction between Tregs & B cells leads to the release of cytokines IL10 and TGF-beta (from B cells & Tregs, respectively). As a result, B-cells are stimulated to produce IgG4. IgG4 binds to allergen, blocking it from binding to IgE on the surface of mast cells.

Thus, one result of immunotherapy may be an increase in a number of regulatory T cells (Tregs) and/or T helper type 1 (Th1), which regulate the immune system and possibly inhibit the allergic response. It may also be that immunotherapy decreases counts of T helper type 2 (Th2) cells, which cells may promote the allergic response. Additionally, Th2 cells may signal, via IL-4 & IL-13 (cytokines), B cells to class switch, changing the Ab production of those B cells to the production of IgE, which amplifies the Th2 response. Under immunotherapy, after a period of time Th1 cells greatly outnumber Th2 cells, which minimizes B cell class-switching and inhibits the immune response. Those cell and molecular species represent potential targets for including, upregulating, and downregulating in allergy therapies.

A principle intended result of immunotherapy is desensitization. Desensitization, broadly, includes that caused by immunotherapy. However, desensitization may also occur naturally, based on the passage of time, a person's experiences, or even unknown causes. The primary mechanism by which desensitization works may be the production of IgG4 specific to the allergen. However, desensitization may also involve the upregulation of Tregs, Th1 cells, or cytokines such as IL10 and TGF-beta. Similarly, desensitization may involve the downregulation of Th2 cells or cytokines such as IL-4 & IL-13. Desensitization may primarily involve IgE competition (e.g., by IgG4 or some other blocking antibody), or desensitization may involve immunomodulation generally, or desensitization may lie on a spectrum between general immunomodulation and IgE blocking, with elements of both involve.

An important insight of the invention is that people who have become desensitized to an allergen may have circulating factors, such as molecular species or cells that are useful to prevent an allergic reaction. Where a subject has been allergic to a specific allergen, but then becomes desensitized, that person's blood, plasma, or serum may be a source of factors, such as molecular species, cells, or fragments thereof that neutralize the allergen, and inhibit its ability to trigger an allergic or anaphylactic response. Those factors may be obtained, isolated, or identified and enriched, made into, or included in a composition that is useful as a therapeutic to treat allergies. The primary effective ingredient of that therapeutic composition may be IgG4 specific to the allergen, or it may be any other factor or material from the person, or it may be other antibodies that block the allergic response, or any of those materials in combination.

To prepare the therapeutic composition, methods of the invention may include taking a sample (e.g., blood, plasma, serum, lymph, mucous, saliva, or any other suitable sample) from desensitized individuals. The sample may include blocking antibodies (e.g., IgG4 or other) that inhibit the allergic response. Methods include administering the blocking antibodies to people may have an allergy. Using those insights, the invention provides methods of allergy treatment.

Methods of the invention include obtaining sample from a person who has become desensitized to an allergy. Any suitable people or population may be sampled. For example, subjects who have undergone oral immunotherapy and become desensitized may represent a suitable population to obtain samples from. Some such subjects may have participated in oral immunotherapy as a cohort in a study sponsored by an institution, or through participation in a treatment program administered by an institution, and in such cases the institution may have a “bank” or “collection” of samples, e.g., blood or plasma or serum samples from those subjects. Such a collection of samples may be surveyed or assayed to identify factors that contribute desensitization. Methods may include analyzing a sample to identify a molecular species that inhibits an allergic response.

In some embodiments, RNA-seq is performed on B cells isolated from the peripheral blood of food allergy desensitized individuals, and each cell's gene expression, splice variants, and heavy and light chain antibody sequences are characterized. Blood may be separated into plasma and cellular fractions; plasma stored and later used for allergen-specific immunoglobin concentration measurements, while the cellular fraction may be enriched for B cells prior to FACS. CD19+ B cells may be sorted exclusively based on immunoglobulin surface expression, but with an emphasis on IgE, IgG4 or other Ig-producing B cell capture. Isotype identity may be determined from scRNA-seq. B cell capture by such methods avoid stringent requirements on FACS gate purity or the need for complex gating schemes. Single cells may be sorted into wells or other fluid partition, e.g., droplets on a microfluidics platform, and processed using a modified version of the Smart1-seq2 protocol. See Picelli, 2014, Full-length RNA-seq from single cells using Smart-seq2, Nat Protocol 9:171-181, incorporated by reference. Sequencing may be performed on an Illumina NextSeq 500 with 2×150 bp reads to an average depth of 1-2 million reads per cell. Sequencing reads may be aligned and assembled to produce a gene expression count table and reconstruct antibody heavy and light chains, respectively. Using software such as STAR for alignment also facilitates the assessment of splicing within single cells. See Dobin, 2013, STAR: ultrafast universal RNA-seq aligner, Bioinformatics 29:15-21, incorporated by reference. Cells may be stringently filtered to remove those of low quality, putative basophils, and those lacking a single productive heavy and light chain. Isotype identity of each cell may be determined by its productive heavy chain assembly, which avoids misclassification of isotype based on FACS immunoglobulin surface staining. From such sequences, the sequences of antibodies such as IgG4 and/or IgE may be determined. Such sequences could be cloned and expressed recombinantly. See Dodev, 2014, A tool kit for rapid cloning and expression of recombinant antibodies, Scientific Reports 4:5885, incorporated by reference.

Methods of making and purifying antibodies are known in the art and were developed by 1980s as described Harlow and Lane, 1988, Antibodies: A Laboratory Manual, CSHP, Incorporated by reference. Antibodies (e.g., IgG4 and/or IgE) may be isolated or purified using hybridoma technology, wherein isolated B lymphocytes in suspension are fused with myeloma cells from the same species to create monoclonal hybrid cell lines that are virtually immortal while still retaining their antibody-producing abilities. See Harlow and Lane, 1988, Antibodies: A Laboratory Manual, Cold Spring Harbor Laboratory, incorporated by reference. Such hybridomas may be stored frozen and cultured as needed to produce the specific monoclonal antibody. Such monoclonal antibodies may be deployed therapeutically in methods of the invention. Those immunoglobins exhibit single-epitope specificity and the hybridoma clone cultures provide an unchanging supply over many years. Hybridoma clones may be grown in cell culture for collection of antibodies from the supernatant or grown in the peritoneal cavity of a mouse for collection from ascitic fluid.

Whether by recombinant cloning and expression or by hybridoma technology, immunoglobins from desensitized subject may be provided for use in a therapeutic composition. Methods of the disclosure may include preparing a therapy that includes a molecular species such as an antibody (e.g., an IgG4), a fragment thereof, a cytokine, or a cell, or a fragment thereof, that inhibits an allergic response.

The molecular species (e.g., antibody) may then be prepared for therapeutic delivery. Antibody therapeutics may be given systemically by intravenous (IV) injection or by intramuscular (IM) and subcutaneous (SC) injection modes. Preferably the therapeutic is formulated at a suitably high stable concentration with parameters such as viscosity optimized for a delivery route. For example, viscosity maybe tuned to match a particular syringe or autoinjector. The formulation may comprise an aqueous solution or suspension with suitable buffers and optionally any other excipients to mitigate undesirable protein instability. Example excipients include fillers, extenders, diluents, solvents, preservatives, absorption enhancers and sustained release matrices. Buffers and excipients that are FDA approved for formulation of antibodies are generally known by those of skill in the art.

Once the therapeutic composition has been formulated, it may be provided for delivery to a patient who is potentially susceptible to an allergic reaction. For example, the composition may be packaged, e.g., in a bottle, vial, syringe, autoinjector, reservoir for an autoinjector or other device, or IV bag. The composition may be stored, e.g., in a cooler or freezer, or carried to a clinical setting for delivery. The composition may be packaged, e.g., in dry ice, and shipped to a hospital or other clinical setting for administration to a subject by a clinical professional.

One important embodiment of aspects of the invention includes methods of allergy treatment that include obtaining sample from a person who has become desensitized to an allergy, analyzing the sample to identify a molecular species that inhibits an allergic response, preparing a therapeutic composition that includes the molecular species, and storing the therapeutic composition for later use. Every embodiment of methods of this disclosure may involve preparing a therapeutic composition and storing it for later use. For example, methods of the disclosure may include freezing the therapeutic composition or otherwise preserving and storing the composition. A therapeutic composition may be, for example, dried or lyophilized, e.g., in jars, bottles, or vials, and may optionally be packaged with instructions to aid in later re-constitution or rehydration. It is important to note that any method of this disclosure may be understood, stated, or restated to not include any step of administering the therapy to a patient susceptible to the allergic response. Instead, any statement or description of a method may be understood to mean and include versions of the methods that include making or preparing a therapeutic composition and packaging or storing those compositions, e.g., in a freezer, for subsequent use or distribution.

One important insight of the disclosure is that the composition includes one or more ingredients, such as molecular species, cells, fragment of cells, blood components, others, or various combinations thereof that have been obtain obtained from a patient who has been desensitized to an allergy. Typically, the patient will have an allergy to a specific allergen. The antigen or epitope of the allergen implicated in the patient's allergic responses may be known, and the composition includes ingredients that attenuate the allergic response to that allergen, e.g., to the particular antigen or epitope involved in the allergic response. That effective ingredient may be antibody against that allergen, such as a blocking antibody, i.e., and antibody that blocks the ability of that allergen to bind to, and cross-link, complexes of IgE and Fc receptor. For example, in some preferred embodiments, the effective ingredient is an IgG4 immunoglobin specific to the allergen. An insight of the invention is that the desensitized patient has done an in vivo epitope discovery assays and produced blocking antibodies or other cell or molecular species against the allergen. Helpfully, there may be a match between the epitope or antigen implicated in the allergy to which the subject is desensitized and the epitope or antigen that is implicated in the allergy that is treated by the composition of the invention.

As discussed, the effective ingredient may include a blocking antibody such as an IgG4 specific to the allergen. However, other cell and molecular species may be included. For example, immune cells or fragments of cells may be included. Because desensitization may also involve the upregulation of Tregs or Th1 cells, those cells may be effective or useful to include in a composition. Because the beneficial effects of those cells on attenuating an allergic response may arise from cell surface proteins or cellular/cytoplasmic contents of those cells (e.g., proteins, sugars, lipids, nucleic acids, salts, or combinations or complexes thereof), it may be beneficial to include fragments or remnants of broken or ruptured cells such as those. As it is understood that desensitization may be mediated by other molecular species such as histamine or cytokines such as IL-4R, IL-4, IL-13, IL-33, IL-9, IL-10, or IL-5 or TGF-beta, those ingredients may be included in therapeutic compositions of the invention. Similarly, as desensitization may involve the downregulation of Th2 cells or cytokines such as IL-4 & IL-13, therapeutic compositions of the invention may include molecular species such as antibodies that bind to, sequester, block, or neutralize and of those species. For example, therapeutic compositions of the invention may include opsonins specific to Th2 cells that mark those cells for destruction. The full complement of ingredients from the desensitized subject may be fully identified, or it may be taken and used or included in a therapeutic composition even if all ingredients are not fully known or quantified. Desensitization may primarily involve IgE competition (i.e., blocking antibodies such as IgG4 or some other blocking antibody), or desensitization may involve immunomodulation generally, or desensitization may lie on a spectrum between general immunomodulation and IgE blocking, with elements of both involve. Because an allergic reaction involves mast cells or basophils presenting IgE-bound Fc receptors that bind to and are cross-linked by IgE, compositions may include an antibody fragment, such as an IgE fragment, that may bind an allergen but that does not bind and cross-link Fc receptors.

Those techniques, insights, and formulations described here may be used to address cases of allergy where the causative antigen or epitope apparently changes over time. There may be some cases in which a subject presents with an allergy. Testing may reveal that a particular epitope of an allergen is implicated in the allergic reaction, i.e., because the subject is making abundant IgE specific to the particular epitope. However, it may be found that the same subject apparently has the same allergy a time later (e.g., months or years) but testing at the later point in time may reveal that the subject is, in fact, reacting to a different epitope of the same allergen. Those circumstances may arise naturally, as a product of the subject's natural body and immune functions. Those circumstances may arise for reasons that are not known. Those circumstances may arise due to a therapeutic intervention such as oral immunotherapy. For example, it may be that immunotherapy effectively promotes production of blocking antibodies in the subject such as IgG4 specific to the epitope. Without being bound by any mechanism, it may be that those blocking antibodies then present a selective force against the IgE specific to the particular epitope. For example, the blocking antibodies may interfere with, or mark for destruction, B cells that make the IgE specific to the particular epitope. Such circumstances may permit abundant production of a second IgE specific to a second epitope. In such cases, the subject could continue to exhibit an anaphylactic response to the allergen because the second IgE is bound to and cross-linked by the second epitope. It may be in such circumstances that the subject has undergone a natural version of an epitope discovery assay, and the second IgE may potentially present new information about new molecular targets suitable for targeting in an allergy therapy.

Here, that the molecular targets are new may mean that those targets were not yet known to any particular clinician or researcher or not yet identified in the literature. An insight of the invention is that those targets represent a potentially valuable path to the creation of useful or effective therapies. Under those circumstances, the inventions provide methods of treating allergy that include detecting—from a subject with an allergy—IgE specific to an epitope of an allergen and—when B cells from the subject have produced a second IgE specific to a second epitope of the allergen—identifying a sequence or structure of the second epitope or a paratope of the second IgE. These methods may be useful where a subject has been treated to attenuate allergic response to the epitope. The identified sequence of the second epitope may be used to determine a therapy for use in an additional, subsequent treatment for allergic response or prophylaxis to an allergic exposure

These methods may include assays discussed above for isolating B cells and sequencing or cloning IgE (e.g., at first and second times) from the subject. In some embodiments, the sequence of the IgE is used to model and predict and thereby discover the sequence or structure of the second epitope. Any suitable method may be used to discover a sequence or a structure of an epitope when a new antibody is discovered. For example, once an antibody is discovered, its sequence and structure may be determined by, e.g., nucleic acid sequencing (e.g., RNA-Seq of transcripts from a B cell), protein sequencing, crystallography, mass spectrometry, and preferably by combinations of the foregoing such as x-ray crystallography, peptide sequencing, and mass spectrometry. As is known in the art, protein structure may be stored in a file format known as protein data bank format. Here, methods may include modelling the structure of the first and/or the second IgE in protein data bank format.

There are software programs known in the art useful to discover or predict sequence or structure of an epitope from a known antibody structure. For example, ClusPro is a software product that includes a software product called PIPER that can predict an epitope structure from an antibody structure. See Kozakov, 2017, The ClusPro web server for protein-protein docking, Nat Protoc 12(2):255-278 and Kozakov, 2006, PIPER: an FFT-based protein docking program with pairwise potentials, Proteins 65:392-406, both incorporated by reference.

Once the structure of the second epitope is determined, one option is to perform in vitro assays to identify blocking antibodies for the second epitope. It may also be that discovery of the second epitope reveals that the subject is reacting to a known epitope with an existing therapeutic. For example, discovery of the second epitope may reveal that the subject is a good candidate for immunotherapy, e.g., oral immunotherapy. For example, it maybe that the subject first reacted to a rare or unknown epitope but that after time, the subject is now exhibiting the same allergic response but to a different, second epitope. It may be found that the subject is now reacting to a relatively common or ubiquitous epitope of the allergen and is now a good candidate for oral immunotherapy. An important insight of these embodiments is that persistent or clinically difficult allergy is not insurmountable. Discovery that the subject is reacting to a second epitope may present opportunities and options for therapies. Methods may include preparing a therapeutic composition that prevents the second epitope from promoting IgE cross-linking in vivo. Once the second epitope is known, the therapeutic composition may be made with a molecular blocker that interferes with in vivo binding of IgE to the allergen (e.g., an IgG4 that binds the second epitope). The therapeutic composition may include any of those ingredients discussed above, e.g., any of those molecular species or cells or fragments thereof or any combination thereof.

Once the second epitope is discovered, the sequence or structure of the second epitope may be added to a database of therapeutic targets. That is the repertoire of knowledge of allergens may be expanded to improve a range of treatment options and discovery opportunities for future patients.

Once the second epitope is discovered, it may be used to create blocking antibodies against itself. Antibodies can be produced in animals, i.e., by immunization an animal with the second epitope. Once the sequence of the second epitope is known, it can be cloned, e.g., into yeast or bacteria, and grown up in bulk to form a protein product that primarily includes the second epitope for use in animal immunization to raise blocking antibodies. The protein product can be harvested from the growth vector and inoculated into animals (e.g., mice) to cause them to grow antibodies against the second epitope. Those antibodies may be harvested and optionally sequenced and/or cloned via hybridoma technology for further expansion, e.g., followed by isolation for use in a therapeutic composition. From those approaches, a subject with an allergy for whom the apparently implicated epitope has changed over time from a first epitope to a second epitope may be used as a source of information or sample for epitope discovery and for input into a laboratory cloning and expansion method to create a therapeutic against the second epitope.

In another approach when the second IgE is known or identified, anti-IgE antibody is made for use in a therapeutic composition. A therapeutic composition is prepared (according to methods for formulations discussed above) and stored or administered to a person suspected of being at risk for allergy including optionally the subject. In the person, the anti-IgE antibody may bind to copies the second IgE and prevent that IgE from binding, and being cross-linked by, the second epitope. Thus, methods may include producing antibodies (e.g., monoclonal antibodies) such as IgG4 or other specific to one of the first and second epitopes or specific to the first or second IgE.

Throughout the present description it is understood that methods of the inventions may be used to respond to, study, or treat allergies to any any allergen, including but not limited to the following allergens; Ambrosia artemisiifolia (short ragweed) antigen E (Amb a 1); Ambrosia artemisiifolia (short ragweed) antigen K (Amb a 2); Ambrosia artemisiifolia (short ragweed) Ra3 antigen (Amb a 3); Ambrosia artemisiifolia (short ragweed) Ra5 antigen (Amb a 5); Ambrosia artemisiifolia (short ragweed) Ra6 antigen (Amb a 6); Ambrosia artemisiifolia (short ragweed) Ra7 antigen (Amb a 7); Ambrosia trifida (giant ragweed) Ra5G antigen (Amb t 5); Artemisia vulgaris (mugwort) antigen (Art v 1); Artemisia vulgaris (mugwort) antigen (Art v 2); Helianthus annuus (sunflower) antigen (Hel a 1); Helianthus annuus (sunflower) profilin (Hel a 2); Mercurialis annua (annual mercury) profilin (Mer a 1); Cynodon dactylon (Bermuda grass) antigen (Cyn d 1); Cynodon dactylon (Bermuda grass) antigen (Cyn d 7); Cynodon dactylon (Bermuda grass) profilin (Cyn d 12); Dactylis glomerata (orchard grass) AgDg1 antigen (Dac g 1); Dactylis glomerata (orchard grass) antigen (Dac g 2); Dactylis glomerata (orchard grass) antigen (Dac g 3); Dactylis glomerata (orchard grass) antigen (Dac g 5); Holcus lanatus (velvet grass) antigen (Hol l 1); Lolium perenne (rye grass) group I antigen (Lol p 1); Lolium perenne (rye grass) group II antigen (Lol p 2); Lolium perenne (rye grass) group III antigen (Lol p 3); Lolium perenne (rye grass) group IX antigen (Lol p 5); Lolium perenne (rye grass) antigen (Lol p Ib); Lolium perenne (rye grass) trypsin (Lol p 11); Phalaris aquatica (canary grass) antigen (Pha a 1); Phleum pratense (timothy grass) antigen (Phl p 1); Phleum pratense (timothy grass) antigen (Phl p 2); Phleum pratense (timothy grass) antigen (Phl p 4); Phleum pratense (timothy grass) antigen Ag 25 (Phl p 5); Phleum pratense (timothy grass) antigen (Phl p 6); Phleum pratense (timothy grass) profilin (Phl p 12); Phleum pratense (timothy grass) polygalacturonase (Phl p 13); Poa pratensis (Kentucky blue grass) group I antigen (Poa p 1); Poa pratensis (Kentucky blue grass) antigen (Poa p 5); Sorghum halepense (Johnson grass) antigen (Sor h 1); Alnus glutinosa (alder) antigen (Aln g 1); Betula verrucosa (birch) antigen (Bet v 1); Betula verrucosa (birch) profilin (Bet v 2); Betula verrucosa (birch) antigen (Bet v 3); Betula verrucosa (birch) antigen (Bet v 4); Betula verrucosa (birch) isoflavone reductase homologue (Bet v 5); Betula verrucosa (birch) cyclophilin (Bet v 7); Carpinus betulus (hornbeam) antigen (Car b 1); Castanea sativa (chestnut) Bet v 1 homologue (Cas s 1); Castanea sativa (chestnut) chitinase (Cas s 5); Corylus avelana (hazel) antigen (Cor a 1); Quercus alba (white oak) antigen (Que a 1); Cryptomeria japonica (sugi) antigen (Cry j 1); Cryptomeria japonica (sugi) antigen (Cry j 2); Juniperus ashei (mountain cedar) antigen (Jun a 1); Juniperus ashei (mountain cedar) antigen (Jun a 3); Juniperus oxycedrus (prickly juniper) calmodulin-like antigen (Jun o 2); Juniperus sabinoides (mountain cedar) antigen (Jun s 1); Juniperus virginiana (eastern red cedar) antigen (Jun v 1); Fraxinus excelsior (ash) antigen (Fra e 1); Ligustrum vulgare (privet) antigen (Lig v 1); Olea europea (olive) antigen (Ole e 1); Olea europea (olive) profilin (Ole e 2); Olea europea (olive) antigen (Ole e 3); Olea europea (olive) antigen (Ole e 4); Olea europea (olive) superoxide dismutase (Ole e 5); Olea europea (olive) antigen (Ole e 6); Syringa vulgaris (lilac) antigen (Syr v 1); Acarus siro (mite) fatty acid-binding protein (Aca s 13); Blomia tropicalis (mite) antigen (Blo t 5); Blomia tropicalis (mite) Bt11a antigen (Blo t 12); Blomia tropicalis (mite) Bt6 fatty acid-binding protein (Blo t); Dermatophagoides pteronyssinus (mite) antigen P1 (Der p 1); Dermatophagoides pteronyssinus (mite) antigen (Der p 2); Dermatophagoides pteronyssinus (mite) trypsin (Der p 3); Dermatophagoides pteronyssinus (mite) amylase (Der p 4); Dermatophagoides pteronyssinus (mite) antigen (Der p 5); Dermatophagoides pteronyssinus (mite) chymotrypsin (Der p 6); Dermatophagoides pteronyssinus (mite) antigen (Der p 7); Dermatophagoides pteronyssinus (mite) glutathione transferase (Der p 8); Dermatophagoides pteronyssinus (mite) collagenolytic serine prot (Der p 9); Dermatophagoides pteronyssinus (mite) tropomyosin (Der p 10); Dermatophagoides pteronyssinus (mite) apolipophorin like p (Der p 14); Dermatophagoides microceras (mite) antigen (Der m 1); Dermatophagoides farinae (mite) antigen (Der f 1); Dermatophagoides farinae (mite) antigen (Der f 2); Dermatophagoides farinae (mite) antigen (Der f 3); Dermatophagoides farinae (mite) tropomyosin (Der f 10); Dermatophagoides farinae (mite) paramyosin (Der f 11); Dermatophagoides farinae (mite) Mag 3, apolipophorin (Der f 14); Euroglyphus maynei (mite) apolipophorin (Eur m 14); Lepidoglyphus destructor (storage mite) antigen (Lep d 2.0101); Lepidoglyphus destructor (storage mite) antigen (Lep d 2.0102); Bos domesticus (cow) Ag3, lipocalin (Bos d 2); Bos domesticus (cow) alpha-lactalbumin (Bos d 4); Bos domesticus (cow) beta-lactalbumin (Bos d 5); Bos domesticus (cow) serum albumin (Bos d 6); Bos domesticus (cow) immunoglobulin (Bos d 7); Bos domesticus (cow) casein (Bos d 8); Canis familiaris (dog) antigen (Can f 1); Canis familiaris (dog) antigen (Can f 2); Canis familiaris (dog) albumin (Can f ?); Equus caballus (horse) lipocalin (Equ c 1); Equus caballus (horse) lipocalin (Equ c 2); Felis domesticus (cat) cat-1 antigen (Fel d 1); Mus musculus (mouse) MUP antigen (Mus m 1); Rattus norvegius (rat) antigen (Rat n 1); Alternaria alternata (fungus) antigen (Alt a 1); Alternaria alternata (fungus) antigen (Alt a 2); Alternaria alternata (fungus) heat shock protein (Alt a 3); Alternaria alternata (fungus) ribosomal protein (Alt a 6); Alternaria alternata (fungus) YCP4 protein (Alt a 7); Alternaria alternata (fungus) aldehyde dehydrogenase (Alt a 10); Alternaria alternata (fungus) enloase (Alt a 11); Alternaria alternata (fungus) acid ribosomal protein P1 (Alt a 12); Cladosporium herbarum (fungus) antigen (Cla h 1); Cladosporium herbarum (fungus) antigen (Cla h 2); Cladosporium herbarum (fungus) aldehyde dehydrogenase (Cla h 3); Cladosporium herbarum (fungus) ribosomal protein); Cladosporium herbarum (fungus) YCP4 protein (Cla h 5); Cladosporium herbarum (fungus) enolase (Cla h 6); Cladosporium herbarum (fungus) acid ribosomal protein P1 (Cla h 12); Aspergillus flavus (fungus) alkaline serine proteinase (Asp fl 13); Aspergillus fumigatus (fungus) antigen (Asp f 1); Aspergillus fumigatus (fungus) antigen (Asp f 2); Aspergillus fumigatus (fungus) peroxisomal protein (Asp f 3); Aspergillus fumigatus (fungus) antigen (Asp f 4); Aspergillus fumigatus (fungus) metalloprotease (Asp f 5); Aspergillus fumigatus (fungus) Mn superoxide dismutase (Asp f 6); Aspergillus fumigatus (fungus) antigen (Asp f 7); Aspergillus fumigatus (fungus) ribosomal protein P2 (Asp f 8); Aspergillus fumigatus (fungus) antigen (Asp f 9); Aspergillus fumigatus (fungus) aspartis protease (Asp f 10); Aspergillus fumigatus (fungus) peptidyl-prolyl isomerase (Asp f 11); Aspergillus fumigatus (fungus) heat shock protein P70 (Asp f 12); Aspergillus fumigatus (fungus) alkaline serine proteinase (Asp f 13); Aspergillus fumigatus (fungus) antigen (Asp f 15); Aspergillus fumigatus (fungus) antigen (Asp f 16); Aspergillus fumigatus (fungus) antigen (Asp f 17); Aspergillus fumigatus (fungus) vacuolar serine (Asp f 18); Aspergillus niger (fungus) beta-xylosidase (Asp n 14); Aspergillus niger (fungus) antigen (Asp n 18); Aspergillus niger (fungus) vacuolar serine proteinase; Aspergillus oryzae (fungus) TAKA-amylase A (Asp o 2); Aspergillus oryzae (fungus) alkaline serine proteinase (Asp o 13); Penicillium brevicompactum (fungus) alkaline serine proteinase (Pen b 13); Penicillium citrinum (fungus) heat shock protein P70 (Pen c 1); Penicillium citrinum (fungus) peroxisomal membrane protein (Pen c 3); Penicillium citrinum (fungus) alkaline serine proteinase (Pen c 13); Penicillium notatum (fungus) N-acetyl glucosaminidase (Pen n 1); Penicillium notatum (fungus) alkaline serine proteinase (Pen n 13); Penicillium notatum (fungus) vacuolar serine proteinase (Pen n 18); Penicillium oxalicum (fungus) vacuolar serine proteinase (Pen o 18); Trichophyton rubrum (fungus) antigen (Tri r 2); Trichophyton rubrum (fungus) serine protease (Tri r 4); Trichophyton tonsurans (fungus) antigen (Tri t 1); Trichophyton tonsurans (fungus) serine protease (Tri t 4); Candida albicans (fungus) antigen (Cand a 1); Candida boidinii (fungus) antigen (Cand b 2); Malassezia furfur (fungus) antigen (Mal f 1); Malassezia furfur (fungus) MF1 peroxisomal membrane protein (Mal f 2); Malassezia furfur (fungus) MF2 peroxisomal membrane protein (Mal f 3); Malassezia furfur (fungus) antigen (Mal f 4); Malassezia furfur (fungus) antigen (Mal f 5); Malassezia furfur (fungus) cyclophilin homologue (Mal f 6); Psilocybe cubensis (fungus) antigen (Psi c 1); Psilocybe cubensis (fungus) cyclophilin (Psi c 2); Coprinus comatus (shaggy cap) antigen (Cop c 1); Coprinus comatus (shaggy cap) antigen (Cop c 2); Coprinus comatus (shaggy cap) antigen (Cop c 3); Coprinus comatus (shaggy cap) antigen (Cop c 5); Coprinus comatus (shaggy cap) antigen (Cop c 7); Aedes aegyptii (mosquito) apyrase (Aed a 1); Aedes aegyptii (mosquito) antigen (Aed a 2); Apis mellifera (honey bee) phospholipase A2 (Api m 1); Apis mellifera (honey bee) hyaluronidase (Api m 2); Apis mellifera (honey bee) melittin (Api m 4); Apis mellifera (honey bee) antigen (Api m 6); Bombus pennsylvanicus (bumble bee) phospholipase (Bom p 1); Bombus pennsylvanicus (bumble bee) protease (Bom p 4); Blattella germanica (German cockroach) Bd90k (Bla g 1); Blattella germanica (German cockroach) aspartic protease (Bla g 2); Blattella germanica (German cockroach) calycin (Bla g 4); Blattella germanica (German cockroach) glutathione transferase (Bla g 5); Blattella germanica (German cockroach) troponin C (Bla g 6); Periplaneta americana (American cockroach) Cr-PII (Per a 1); Periplaneta americana (American cockroach) Cr-PI (Per a 3); Periplaneta americana (American cockroach) tropomyosin (Per a 7); Chironomus thummi thummi (midge) hemoglobin (Chi t 1-9); Chironomus thummi thummi (midge) component III (Chi t 1.01); Chironomus thummi thummi (midge) component IV (Chi t 1.02); Chironomus thummi thummi (midge) component I (Chi t 2.0101); Chironomus thummi thummi (midge) component IA (Chi t 2.0102); Chironomus thummi thummi (midge) component II-beta (Chi t 3); Chironomus thummi thummi (midge) component IIIA (Chi t 4); Chironomus thummi thummi (midge) component VI (Chi t 5); Chironomus thummi thummi (midge) component VIIA (Chi t 6.01); Chironomus thummi thummi (midge) component IX (Chi t 6.02); Chironomus thummi thummi (midge) component VIIB (Chi t 7); Chironomus thummi thummi (midge) component VIII (Chi t 8); Chironomus thummi thummi (midge) component X (Chi t 9); Dolichovespula maculata (white face hornet) phospholipase (Dol m 1); Dolichovespula maculata (white face hornet) hyaluronidase (Dol m 2); Dolichovespula maculata (white face hornet) antigen 5 (Dol m 5); Dolichovespula arenaria (yellow hornet) antigen 5 (Dol a 5); Polistes annularies (wasp) phospholipase A1 (Pol a 1); Polistes annularies (wasp) hyaluronidase (Pol a 2); Polistes annularies (wasp) antigen 5 (Pol a 5); Polistes dominulus (Mediterranean paper wasp) antigen (Pol d 1); Polistes dominulus (Mediterranean paper wasp) serine protease (Pol d 4); Polistes dominulus (Mediterranean paper wasp) antigen (Pol d 5); Polistes exclamans (wasp) phospholipase A1 (Pol e 1); Polistes exclamans (wasp) antigen 5 (Pol e 5); Polistes fuscatus (wasp) antigen 5 (Pol f 5); Polistes metricus (wasp) antigen 5 (Pol m 5); Vespa crabo (European hornet) phospholipase (Vesp c 1); Vespa crabo (European hornet) antigen 5 (Vesp c 5.0101); Vespa crabo (European hornet) antigen 5 (Vesp c 5.0102); Vespa mandarina (giant Asian hornet) antigen (Vesp m 1.01); Vespa mandarina (giant Asian hornet) antigen (Vesp m 1.02); Vespa mandarina (giant Asian hornet) antigen (Vesp m 5); Vespula flavopilosa (yellowjacket) antigen 5 (Ves f 5); Vespula germanica (yellowjacket) antigen 5 (Ves g 5); Vespula maculifrons (yellowjacket) phospholipase A1 (Ves m 1); Vespula maculifrons (yellowjacket) hyaluronidase (Ves m 2); Vespula maculifrons (yellowjacket) antigen 5 (Ves m 5); Vespula pennsylvanica (yellowjacket) (antigen 5Ves p 5); Vespula squamosa (yellowjacket) antigen 5 (Ves s 5); Vespula vidua (wasp) antigen (Ves vi 5); Vespula vulgaris (yellowjacket) phospholipase A1 (Ves v 1); Vespula vulgaris (yellowjacket) hyaluronidase (Ves v 2); Vespula vulgaris (yellowjacket) antigen 5 (Ves v 5); Myrmecia pilosula (Australian jumper ant) antigen (Myr p 1); Myrmecia pilosula (Australian jumper ant) antigen (Myr p 2); Solenopsis geminata (tropical fire ant) antigen (Sol g 2); Solenopsis geminata (tropical fire ant) antigen (Sol g 4); Solenopsis invicta (fire ant) antigen (Sol i 2); Solenopsis invicta (fire ant) antigen (Sol i 3); Solenopsis invicta (fire ant) antigen (Sol i 4); Solenopsis saevissima (Brazilian fire ant) antigen (Sol s 2); Gadus callarias (cod) allergen M (Gad c 1); Salmo salar (Atlantic salmon) parvalbumin (Sal s 1); Gallus domesticus (chicken) ovomucoid (Gal d 1); Gallus domesticus (chicken) ovalbumin (Gal d 2); Gallus domesticus (chicken) conalbumin; A22 (Gal d 3); Gallus domesticus (chicken) lysozyme (Gal d 4); Gallus domesticus (chicken) serum albumin (Gal d 5); Metapenaeus ensis (shrimp) tropomyosin (Met e 1); Penaeus aztecus (shrimp) tropomyosin (Pen a 1); Penaeus indicus (shrimp) tropomyosin (Pen i 1); Todarodes pacificus (squid) tropomyosin (Tod p 1); Haliotis midae (abalone) antigen (Hal m 1); Apium graveolens (celery) Betv 1 homologue (Api g 1); Apium graveolens (celery) profilin (Api g 4); Apium graveolens (celery) antigen (Api g 5); Brassica juncea (oriental mustard) 2S albumin (Bra j 1); Brassica rapa (turnip) prohevein-like protein (Bar r 2); Hordeum vulgare (barley) BMAI-1 (Hor v 1); Zea mays (maize, corn) lipid transfer protein (Zea m 14); Corylus avellana (hazelnut) Betv 1 homologue (Cor a 1.0401); Malus domestica (apple) Bet v 1 homologue (Mal d 1); Malus domestica (apple) lipid transfer protein (Mal d 3); Pyrus communis (pear) Bet v 1 homologue (Pyr c 1); Pyrus communis (pear) profilin (Pyr c 4); Pyrus communis (pear) isoflavone reductase homologue (Pyr c 5); Oryza sativa (rice) antigen (Ory s 1); Persea americana (avocado) endochitinase (Pers a 1); Prunus armeniaca (apricot) Bet v 1 homologue (Pru ar 1); Prunus armeniaca (apricot) lipid transfer protein (Pru ar 3); Prunus avium (sweet cherry) Bet v 1 homologue (Pru av 1); Prunus avium (sweet cherry) thaumatin homologue (Pru av 2); Prunus avium (sweet cherry) profilin (Pru av 4); Prunus persica (peach) lipid transfer protein (Pru p 3); Sinapis alba (yellow mustard) 2S albumin (Sin a 1); Glycine max (soybean) HPS (Gly m 1.0101); Glycine max (soybean) HPS (Gly m 1.0102); Glycine max (soybean) antigen (Gly m 2); Glycine max (soybean) profilin (Gly m 3); Arachis hypogaea (peanut) vicilin (Ar a h 1); Arachis hypogaea (peanut) (conglutin Ar a h 2); Arachis hypogaea (peanut) glycinin (Ar a h 3); Arachis hypogaea (peanut) glycinin (Ar a h 4); Arachis hypogaea (peanut) (profilin Ar a h 5); Arachis hypogaea (peanut) conglutin homologue (Ar a h 6); Arachis hypogaea (peanut) conglutin homologue (Ar a h 7); Actinidia chinensis (kiwi) cysteine protease (Act c 1); Solanum tuberosum (potato) patatin (Sol t 1); Bertholletia excelsa (Brazil nut) 2S albumin (Ber e 1); Juglans regia (English walnut) 2S albumin (Jug r 1); Juglans regia (English walnut) vicilin (Jug r 2); Ricinus communis (castor bean) 2S albumin (Ric c 1); Anisakis simplex (nematode) antigen (Ani s 1); Anisakis simplex (nematode) paramyosin (Ani s 2); Ascaris suum (worm) antigen (Asc s 1); Aedes aegyptii (mosquito) apyrase (Aed a 1); Aedes aegyptii (mosquito) antigen (Aed a 2); Hevea brasiliensis (rubber) elongation factor (Hev b 1); Hevea brasiliensis (rubber) 1,3-glucanase (Hev b 2); Hevea brasiliensis (rubber) antigen (Hev b 3); Hevea brasiliensis (rubber) component of microhelix protein complex (Hev b 4); Hevea brasiliensis (rubber) antigen (Hev b 5); Hevea brasiliensis (rubber) hevein precursor (Hev b 6.01); Hevea brasiliensis (rubber) hevein (Hev b 6.02); Hevea brasiliensis (rubber) C-terminal fragment antigen (Hev b 6.03); Hevea brasiliensis (rubber) patatin homologue (Hev b 7); Hevea brasiliensis (rubber) profilin (Hev b 8); Hevea brasiliensis (rubber) enolase (Hev b 9); Hevea brasiliensis (rubber) Mn-superoxide dismut (Hev b 10); and Ctenocephalides felis felis (cat flea) antigen (Cte f 1). In addition, an allergen may be a component of a vaccine, such as preservatives (e.g., thimersosal, monosodium glutamate), adjuvants (e.g., aluminum, lipids, nucleic acid, polyethylene glycol), stabilizers (e.g., gelatins), residual cell culture materials (e.g., proteins, nucleic acids, yeast), residual inactivating ingredients (e.g., formaldehyde), and antibiotics. Preferred targets include food allergens such as from nuts, fish, milk, etc., as well as venoms, pollens, dander, latex, fungi, medicines (including antibiotics) and in particular peanut, milk, shellfish, tree nuts, egg, fin fish, wheat, soy, and sesame.

1. A method of preparing an allergy treatment, the method comprising:
obtaining at least one sample from at least one person who has become desensitized to an allergen;
analyzing the sample to identify a molecular species that inhibits an allergic response; and
preparing a therapeutic composition that includes the molecular species.

2. (canceled)

3. The method of claim 1, wherein the molecular species is a monoclonal IgG4 that binds the allergen.

4.-5. (canceled)

6. The method of claim 3, wherein the obtaining step comprises obtaining a plurality of samples and the analyzing step comprises identifying a plurality of different IgG4s specific to the allergen from the respective samples for preparing the therapeutic composition.

7. The method of claim 1, wherein the analyzing step includes sequencing IgG4 transcripts from the sample to determine a sequence of an IgG4 and the preparing step includes creating a monoclonal antibody product comprising the IgG4, wherein the IgG4 binds to an epitope of the allergen that is recognized by an IgE of the person.

8.-13. (canceled)

14. The method of claim 1, wherein the analyzing step comprises performing RNA-Seq on the sample obtained from the at least one person to identify IgG4 transcripts and at least one capture assay for proteins that bind the allergen.

15. The method of claim 1, wherein the sample comprises blood, plasma, or serum from the patient.

16-30. (canceled)

31. The method of claim 3, wherein the preparing step includes growing the monoclonal IgG4 by hybridoma technology.

32. The method of claim 31, wherein the preparing step comprises formulating a therapeutic composition comprising the monoclonal IgG4 in an aqueous suspension with a pharmaceutically acceptable excipient or buffer.

33. The method of claim 6, wherein the obtaining step comprises obtaining at least one sample from a plurality of people desensitized to the allergen.

34. The method of claim 33, wherein prior to providing the sample, the plurality of people were desensitized to the allergen by therapy.

35. The method of claim 6, wherein the obtaining step comprises obtaining a plurality of samples from a single person.

36. The method of claim 1, wherein the molecular species includes a fragment of an IgE that binds an allergen that promotes the allergic response, wherein the fragment does not bind to a high-affinity Fc receptor.

37. The method of claim 36, wherein the analyzing step comprises determining a sequence of the IgE that binds the allergen and the preparing step comprises using sequence information of the IgE to create the IgE fragment.

38. The method of claim 1, wherein the molecular species is an antibody that binds a cytokine that promotes the allergic response.

39. The method of claim 38, wherein the cytokine is one selected from the group consisting of IL-4R, IL-4, IL-13, IL-33, IL-9, and IL-5.

40. The method of claim 1, wherein the molecular species includes one or more of a Treg cell, a Th1 cell, or a fragment thereof.
